(19) United States
(12) Patent Application Publication
(10) Pub. No.: US 2023/0320951 A1
(43) Pub. Date: Oct. 12, 2023

(54) DENTAL PROPHY PASTE

(71) Applicant: Ivoclar Vivadent AG, Schaan (LI)

(72) Inventors: Urs LENDENMANN, Grabs (CH); Janina SCHOPPE, Feldkirch (AT); Ulrich GEITER, Mauren (LI); Philipp BECKER, Sevelen (CH); Marc OELMANN, Buchs (CH)

(21) Appl. No.: 18/043,086

(22) PCT Filed: Aug. 26, 2021
(86) PCT No.: PCT/EP2021/073638

§ 371 Date: Feb. 27, 2023

(30) Foreign Application Priority Data
Aug. 28, 2020 (EP) 20193325.6

(51) Int. Cl. A61K 8/26; A61K 8/96; A61K 8/25; A61K 8/34; A61Q 11/00

(52) U.S. Cl. CPC A61K 8/26; A61K 8/965; A61K 8/25; A61K 8/342; A61Q 11/00; A61K 2800/28

(57) ABSTRACT

Dental polishing and cleaning paste for professional tooth cleaning, which contains 1 to 30 wt.-% water, 25 to 70 wt.-% at least one humectant, 25 to 60 wt.-% at least one abrasive and 0.05 to 2 wt.-% at least one surfactant, in each case relative to the total mass of the polishing and cleaning paste.

The invention relates to compositions which are particularly suitable as dental polishing and cleaning pastes. The pastes have a smooth consistency and are characterized in that they do not dry out or deliquesce in air.

### STATE OF THE ART

Bacterial plaque compromises hard tooth tissue and periodontium. If no protective measures are taken, it can result in demineralization of the teeth, which ultimately leads to caries. A supragingival accumulation of plaque can also lead to periodontal diseases, for example to gingivitis, periodontitis or also to mucositis or peri-implantitis. It is therefore necessary to keep surfaces inside the mouth as free of plaque as possible. This applies both to natural teeth and to restored teeth or those replaced by implants.

In addition to oral hygiene using toothbrush and toothpaste at home, professional tooth cleaning and polishing make an important contribution to the reduction of the biofilm. Professional tooth cleaning comprises the removal of supragingival dental plaque including dental calculus, the removal of stains as well as the smoothing and polishing of rough surfaces.

The polishing and cleaning pastes used for this resemble the usual toothpastes in their composition. However, they are not suitable for daily dental care at home because they are designed for the special requirements of professional tooth cleaning. Prophy pastes are to offer a maximum cleaning action in order to effectively remove stains and deposits. Moreover, after the polishing the surfaces are to be as smooth as possible.

Prophy pastes contain abrasives, such as for example amorphous silicon dioxide, calcium carbonate, calcium phosphate, hydroxyapatite powder, pumice powder, which are suspended in a liquid matrix. The matrix usually consists of water, glycerol, polyethylene glycol (PEG), propylene glycol and similar orally acceptable solvents.

Natural dental enamel is a very hard tissue and therefore is not very critical with regard to the choice of the abrasives. In contrast, the materials used for the restoration of damaged teeth, such as e.g. plastic filling materials and dental metal alloys, have a much lower hardness, and there is therefore the danger that these will be scratched if unsuitable abrasives are chosen. Scratches make it easier for plaque to settle and are therefore not only a cosmetic problem. Other abrasives, such as e.g. amorphous silicas, are harmless in this regard, but have a much lower cleaning action and thus require a substantially longer cleaning time in order to achieve a comparable cleaning result. Stronger stains cannot be removed from the tooth surface with them.

Toothpastes and prophy pastes must have a viscosity high enough that the abrasives do not sediment and the pastes can be dispensed onto toothbrushes or dental cleaning and polishing instruments without running off. Thickening agents are normally used to set the viscosity. These are usually synthetic or natural polymers.

As a rule, for hygiene reasons, dental prophy pastes for professional use are used as individual packs which contain the quantity of product required for a single application, usually 1.5 to 2 g. A disadvantage is that, due to the small quantity of product, the materials dry out quickly once the pack is opened and can then be used only with difficulty. In addition, for reasons of cost, packaging materials are often used which are not completely vapor-tight, with the result that even the packaged materials have only a limited shelf life. To prevent premature drying out, the pastes usually contain humectants. However, many humectants are hygroscopic, with the result that in damp surroundings the products absorb water. The materials are thereby watered down and deliquesce. It can also happen that a liquid film forms on the surface of the material, which is called bleeding, which likewise impairs the processing properties of the paste.

WO 2009/120854 A2 discloses oral care products which contain gel networks. By gel networks is meant lamellar or vesicular solid crystalline phases which contain a fatty amphiphile, surfactants and solvents. For the production of the gel networks, the fatty amphiphile together with a dispersing surfactant is dispersed in the solvent. The dispersion obtained is then mixed with a swelling surfactant. The addition of the swelling surfactant is said to cause the formation of a gel network, which is said to have a thickening effect.

WO 2010/068424 A2 discloses oral care agents which, in addition to a gel network, contain quartz glass with an average particle size of from 3 to 15 μm as abrasive.

### BRIEF DESCRIPTION OF THE INVENTION

The object of the invention is to provide a dental prophy paste, i.e. a dental polishing and cleaning paste, which does not have the disadvantages of known products, which in particular does not dry out prematurely and which has an optimum consistency for the intended application. The pastes are to make an efficient and quick cleaning of natural and artificial tooth surfaces possible, without scratching the surfaces.

According to the invention, this object is achieved by pastes which comprise
1 to 30 wt.-%, preferably 5 to 25 wt.-% and particularly preferably 10 to 20 wt.-% water,
25 to 70 wt.-%, preferably 30 to 60 wt.-% and particularly preferably 30 to 50 wt.-% of at least one humectant,
25 to 60 wt.-%, preferably 30 to 55 wt.-% and particularly preferably 35 to 50 wt.-% of at least one abrasive and
0.05 to 2 wt.-%, preferably 0.07 to 0.5 wt.-% and particularly preferably 0.1 to 0.2 wt.-% of at least one surfactant.

Unless otherwise indicated, all percentages here are relative to the total mass of the paste.

It has surprisingly been found that prophy pastes with the composition indicated can even be stored unpackaged for longer periods, without losing their good processing properties. They retain a smooth consistency, i.e. they do not dry out or bleed and are not watered down by the absorption of ambient humidity. In addition, they make it possible to clean tooth surfaces quickly and gently without scratching restoration materials, which are softer than natural dental enamel.

### DETAILED DESCRIPTION OF THE INVENTION

### Abrasives

The polishing and cleaning pastes according to the invention contain 25 to 60 wt.-%, preferably 30 to 55 wt.-% and particularly preferably 35 to 50 wt.-% at least one abrasive. The abrasive(s) are preferably selected from kaolin, pumice powder and silica. Polishing and cleaning pastes which contain 0 to 15 wt.-% kaolin, 0 to 50 wt.-% pumice powder and/or 0 to 30 wt.-% silica, wherein the total quantity of the abrasives lies in the named ranges, are particularly preferred.

According to the invention, those compositions which contain at least 10 wt.-%, preferably 10 to 50 wt.-% and particularly preferably 25 to 35 wt.-% kaolin, relative to the total quantity of abrasive, are quite particularly preferred.

On the one hand, dental polishing and cleaning pastes are to clean efficiently and, on the other hand, damage the tooth surface as little as possible. Pumice powder is characterized by a good cleaning action but, because of its strongly abrasive properties, can easily lead to the scratching of tooth surfaces. This is the case in particular for dental restoration materials made of plastic. In contrast, fumed and in particular precipitated silicas have an amorphous structure and consist of rounded particles which scarcely scratch, but for that reason have only a low cleaning power and, in the case of professional tooth cleaning, remove stains less quickly and efficiently.

According to the invention, it has surprisingly been found that kaolin powder is very well suited to the cleaning and polishing of teeth, and makes an efficient and nevertheless gentle cleaning of natural and artificial tooth surfaces possible.

According to the invention, kaolin (CAS No. 1332-58-7) that is as pure as possible is preferably used, particularly preferably kaolin with a kaolin content of more than 90 wt.-%, quite particularly preferably with a kaolin content of more than 98 wt.-% and most preferably approx. 99 wt.-%. The kaolin preferably contains less than 1 wt.-% crystalline silicon dioxide (quartz). According to a particularly preferred embodiment, pharmaceutical-grade kaolin is used.

Furthermore, kaolin powder which has an average particle size (D50) of from 1 to 20 μm, particularly preferably from 2 to 15 μm and quite particularly preferably from 3 to 10 μm, is preferred. In addition, kaolin powders with as small as possible a proportion of coarse-grained particles are preferred. The proportion of coarse-grained particles is characterized by the D90 value. Kaolin powders with a particle size (D90) of less than 30 μm, preferably less than 20 μm and particularly preferably less than 15 μm are particularly suitable according to the invention. Powders with D50 and D90 values in the named ranges make a very gentle cleaning of tooth surfaces possible without scratching restoration materials.

Unless otherwise indicated, the particle-size distribution of powders is measured using a laser diffraction particle size analyzer. The D50 value characterizes the average particle size in the volume distribution and the D90 characterizes the coarser proportion of the powders. D50 means that half of the powder volume is larger than the value indicated and D90 means that only 10% of the powder volume is larger than the value indicated.

Unless otherwise indicated, all particle sizes are volumetric diameters, wherein the particle-size determination in the range of from 0.1 μm to 1000 μm is preferably effected by means of static light scattering, for example using an LA-960 static laser scattering particle size analyzer (Horiba, Japan). Here, a laser diode with a wavelength of 655 nm and an LED with a wavelength of 405 nm are used as light sources. The use of two light sources with different wavelengths makes it possible to measure the entire particle-size distribution of a sample in only one measurement pass, wherein the measurement is carried out as a wet measurement. For this, a 0.1 to 0.5% aqueous dispersion of the filler is prepared and the scattered light thereof is measured in a flow cell. The scattered-light analysis for calculating particle size and particle-size distribution is effected in accordance with the Mie theory according to DIN/ISO 13320. The measurement of the particle size in the range of from 5 nm to 0.1 μm is preferably effected by dynamic light scattering (DLS) of aqueous particle dispersions, preferably with an He-Ne laser with a wavelength of 633 nm, at a scattering angle of 90° and at 25° C., e.g. with a Malvern Zetasizer Nano ZS (Malvern Instruments, Malvern UK).

The prophy pastes according to the invention preferably contain kaolin powder with a D50 value of from 1 to 20 μm and a D90 value below 30 μm, preferably a D50 value of from 2 to 15 μm and a D90 value below 20 μm and particularly preferably with a D50 value of from 3 to 10 μm and a D90 value below 15 μm.

In addition to kaolin, the polishing and cleaning pastes according to the invention can additionally contain further abrasives. Inorganic powders such as calcium carbonate, calcium phosphate, aluminum oxide, aluminum oxyhydroxyhydrate [AlO(OH).H₂O] are particularly suitable as further abrasives. Pumice stone powder as well as synthetic and natural modifications of silicon dioxide are particularly preferred. Diatomaceous earth is a preferred, naturally occurring form of silicon dioxide. Fumed and in particular precipitated silicas are preferred examples of synthetic silicon dioxide modifications. Precipitated silicas with an average particle size (D50) of from 5 to 15 μm and a D99 value <30 μm, and particularly preferably <25 μm, are particularly preferred.

Mixtures of kaolin powder and silica are preferred according to the invention, quite particularly mixtures of kaolin powder and precipitated silica (INCI name: hydrated silica). It has been found that a good polish of the teeth can be achieved significantly more quickly with pastes which contain a mixture of silica and kaolin than with pastes which contain exclusively amorphous silicas. Nevertheless, the pastes are also gentle to similar degrees with respect to dental plastic materials. Mixtures which contain 20 to 40 wt.-% kaolin, particularly preferably 24 to 36 wt.-% and most preferably 26 to 34 wt.-%, relative to the total quantity of kaolin and silica, are quite particularly preferred.

Prophy pastes with higher abrasiveness can be prepared through the addition of pumice stone powder. Pumice stone is a porous vitreous volcanic rock. In chemical terms it is an amorphous aluminum silicate with a Mohs hardness of 6. Pumice stone powder, also called pumice powder for short, is a finely ground powder made of pumice stone. Since it has a comparable hardness to dental enamel, pumice powder is well suited to removing deposits from natural or ceramic tooth surfaces. Pumice stone powder with an average particle size (D50) of from 10 to 60 μm, in particular 15 to 50 μm and quite particularly from 15 to 40 μm is preferred according to the invention. In the case of pumice stone powder it is particularly important that the coarsest particles are not too large, since they cause the deepest scratches. The D99 value can be used as a measure for the coarsest particles; this means that 99% of the particles are smaller than the named value. A pumice stone powder with a D99 value <200 μm, particularly preferably <150 μm and quite particularly preferably <120 μm is preferred.

The abrasiveness of the prophy pastes can be set in a targeted manner by mixing different abrasives. Prophy pastes containing pumice stone are particularly suitable for removing stains from the teeth. Pastes which contain silica and pumice stone powder, a mixture of kaolin, silica and pumice stone powder or a mixture of kaolin and silica are preferred. Pastes which contain exclusively kaolin powder and optionally silica as abrasive are particularly preferred, wherein in all named cases precipitated silica is preferred as silica.

### Humectants

The polishing and cleaning agents according to the invention contain 25 to 70 wt.-%, preferably 30 to 60 wt.-% and particularly preferably 30 to 50 wt.-% at least one humectant.

Humectants preferred according to the invention are glycerol, sorbitol, xylitol, isomaltose, erythritol, propylene carbonate, propylene glycol, triethylene glycol and polyethylene glycol (PEG). Particularly preferred humectants are propylene glycol, triethylene glycol, PEG 200 (CAS No. 25322-68-3), in particular PEG 300 (CAS No. 25322-68-3), PEG 400 (CAS No. 25322-68-3), and glycerol, sorbitol and xylitol and mixtures thereof are quite particularly preferred.

Compositions which contain 18 to 50 wt.-%, particularly preferably 25 to 46 wt.-% and quite particularly preferably 30 to 44 wt.-% glycerol are preferred.

Compositions which in addition to glycerol additionally contain 1 to 5 wt.-% xylitol and/or 1 to 5 wt.-% sorbitol are particularly preferred, wherein the total quantity of xylitol and sorbitol preferably lies in the range of from 1 to 5 wt.-%.

The quantity of the humectant(s) is preferably chosen such that the weight ratio of water to the total quantity of humectant(s) lies in the range of from 0.05 to 0.6, particularly preferably from 0.25 to 0.5 and quite particularly preferably in the region from 0.30 to 0.40.

### Surfactants

The prophy pastes according to the invention contain 0.05 to 2 wt.-%, preferably 0.1 to 0.5 wt.-% and particularly preferably 0.1 to 0.2 wt.-% of at least one surfactant, more preferably a cationic, anionic, and/or nonionic surfactant, most preferably a cationic or anionic surfactant.

Preferred anionic surfactants are linear sodium C₁₂₋₁₈ alkyl sulfates; sodium salts of C₁₂₋₁₃ linear alkyl polyglycol ether sulfates containing from 2 to 6 glycol ether groups in the molecule; alkyl-(C₁₂₋₁₆)-benzene sulfonates; linear alkane-(C₁₂₋₁₈)-sulfonates; sulfosuccinic acid mono-alkyl-(C₁₂₋₁₈)-esters; sulfated fatty acid monoglycerides; sulfated fatty acid alkanolamides; sulfoacetic acid alkyl-(C₁₂₋₁₈)-esters; and acyl sarcosides, acyl taurides and acyl isothionates all containing from 8 to 18 carbon atoms in the acyl moiety. Particularly preferred anionic surfactants are sodium lauryl sulfate and sodium lauryl sacrosinate, especially sodium lauroyl sacrosinate.

Nonionic surfactants contain non-charged polar groups and have no charge. Preferred nonionic surfactants are sucrose laurate, sucrose cocoate, sucrose stearate. Lauryl glucoside is particularly preferred.

Preferred cationic surfactants are cocotrimonium chloride, cocotrimonium methosulfate, dodecyltrimethylammonium chloride (laurtrimonium chloride, CAS No. 112-00-5) and laurtrimonium methosulfate. Quaternary amines or tertiary amines are also suitable as cationic surfactants. Preferred quaternary amine surfactants are cetylpyridinium chloride (hexadecylpyridinium chloride, preferably as a monohydrate, CAS No. 6004-24-6), cetyl triammonium bromide, cetyltrimethylammonium bromide (hexadecyltrimethylammonium bromide), cetyltrimethylammonium chloride, dodecyltrimethylammonium bromide, dodecyl-trimethylammonium chloride, octyl trimethyl ammonium bromide, tetradecyltrimethyl-ammonium bromide, tetradecyltrimethylammonium chloride, dodecylethyldimethylammonium bromide, decyltrimethylammonium bromide, octadecyl trimethyl ammonium bromide, octadecyl triethyl ammonium chloride, trimethyl gam ma-dodecyloxy-beta-hydroxypropyl ammonium chloride, stearoalkonium chloride, olealkonium chloride, cetrimonium chloride, alkyl trimethyl ammonium methosulfate, palmitamidopropyl trimethyl chloride, cetrimonium chloride, and alkyl dimethyl benzyl ammonium chloride. A particularly preferred cationic surfactant is olaflur (2,2′-[[3-[(2-hydroxyethyl)octadecylamino]propyl]imino]bis-ethanol, hydrofluoride, CAS No. 6818-37-7).

Particularly preferred cationic surfactants are cetyltrimethylammonium chloride, behenyl-trimethylammonium chloride, cetylpyridinium chloride (hexadecylpyridinium chloride, preferably as a monohydrate, CAS No. 6004-24-6), octyltrimethylammonium chloride, dodecyltrimethylammonium chloride, hexadecyltrimethylammonium bromide, hexadecyl-trimethylammonium chloride, octyldimethylbenzylammonium chloride, decyldimethylbenzyl-ammonium chloride, stearyldimethylbenzylammonium chloride, didodecyldimethylammonium chloride, dioctadecyldimethylammonium chloride, tallowtrimethylammonium chloride, cocotrimethylammonium chloride. Most preferred cationic surfactants are dodecyltrimethyl-ammonium chloride, dodecyltrimethylammonium bromide cetylpyridinium chloride (hexa-decylpyridinium chloride, preferably as a monohydrate, CAS No. 6004-24-6), hexadecyltrimethylammonium bromide, hexadecyltrimethylammonium chloride and olaflur.

Mixtures of two or more of the above surfactants can be used.

According to the present invention, the most preferred surfactants are sodium lauryl sulfate, lauryl glucoside and sodium lauroyl sacrosinate, wherein sodium lauryl sulfate is particularly preferred.

A particularly preferred embodiment of the present invention are prophy pastes comprising 0.1 to 0.2 wt.-% of sodium lauryl sulfate, cetylpyridinium chloride or dodecyltrimethyl-ammonium chloride.

### Thickening Agents

To set the consistency, the prophy pastes according to the invention preferably also contain a thickening agent.

According to one embodiment, the pastes contain as thickening agent at least one natural or synthetic polymer with thickening properties. Preferred polymers are carboxymethyl cellulose, hydroxyethyl cellulose, methyl cellulose, guar gum, carrageenan, xanthan gum as well as homo- or heteropolymers of acrylic acid (carbomers). Carbomers, carboxymethyl cellulose, hydroxyethyl cellulose and xanthan gum are particularly preferred polymers. Carbomers are preferably used together with a neutralizing agent. Preferred neutralizing agents are sodium or potassium hydroxide solution, ammonia, arginine, aminomethyl propanol, tetrahydroxypropyl ethylenediamine, (ethylenedinitrilo)tetra-2-propanol, triethanol-amine, tromethamine, PEG-15 cocamine, diisopropanolamine and triisopropanolamine.

The prophy pastes according to the invention have a relatively low water content. However, many polymeric thickening agents are water-soluble and therefore require a high water content. Carbomers, xanthan gum and hydroxyethyl cellulose are characterized in that they can also be used in anhydrous compositions or those with low water content.

Polymeric thickening agents are preferably used in a quantity of from 0.2 to 2 wt.-%, particularly preferably from 0.4 to 1.5 wt.-% and quite particularly preferably from 0.5 to 1.0 wt.-%.

According to a preferred embodiment, the prophy pastes according to the invention contain at least one fatty component for the thickening. The fatty component is preferably used in a quantity of from 0.5 to 3 wt.-%, particularly preferably 0.7 to 2.2 wt.-% and quite particularly preferably 1 to 1.3 wt.-%. The fatty component brings about a thickening of the paste through interaction with the surfactant(s), water and humectant. The pastes can additionally contain a polymeric thickening agent, but pastes which do not contain any polymeric thickening agents are preferred.

The fatty component and the surfactant are preferably used in a weight ratio of fatty component to surfactant within a range of 20 to 1.4, preferably 12 to 4, and most preferably 10 to 4.

Hydrocarbons that are not miscible with water and organic compounds with hydrocarbon radicals, which in each case contain carbon chains with at least 12 and quite particularly preferably at least 14 carbon atoms, are particularly suitable as fatty component. Hydrocarbons and hydrocarbon radicals which contain carbon chains with 12 to 30, particularly preferably 12 to 28, quite particularly preferably 14 to 24 carbon atoms and most preferably with 16 to 22 carbon atoms, are preferred. Preferred fatty components are fatty alcohols. Fatty alcohols with a chain length of from 12 to 22 and in particular 14 to 22 carbon atoms are quite particularly preferred. Fatty alcohols with 16 to 18 carbon atoms are the most preferred. Quite particularly preferred fatty alcohols are lauryl alcohol and in particular cetyl alcohol and stearyl alcohol. Mixtures of different fatty components and in particular different fatty alcohols can also be used. A preferred mixture is a mixture of cetyl and stearyl alcohol, which is also called cetyl stearyl alcohol below, particularly preferably an approx. 1:1 mixture.

The fatty component is preferably solid at room temperature and particularly preferably has a melting point of 24° C., quite particularly preferably >40° C. Fatty components with a melting point of from 45 to 60° C. are most preferred.

The fatty components preferred according to the invention are characterized in particular in that they also have excellent thickening properties in the case of low water content.

### Metal Oxides

The prophy pastes according to the invention preferably contain at least one metal oxide. The metal oxide(s) are preferably selected from the oxides of the metals of group 4 of the periodic table of the elements and the oxides of tetravalent metals (Me^{IV}O₂), wherein mixed oxides are preferably excluded. Oxides of zirconium and titanium are particularly preferred.

The oxides of tetravalent metals (Me^{IV}O₂) are particularly preferred, zirconium oxide and in particular titanium dioxide are quite particularly preferred. Anatase is the most preferred. Anatase is one of the three TiO₂ modifications. The other two modifications are rutile and brookite. The metal oxides are preferably not surface-modified. The named metal compounds are characterized by an extremely polar surface.

Surprisingly, it has been found that the addition of the named metal oxides can greatly reduce or even completely prevent the formation of a liquid film on the surface of the pastes, i.e. bleeding of the pastes, even in the case of storage at 40° C. and 75% RH. The pastes remain homogeneous even with changing ambient humidity. This is true in particular for pastes which contain a fatty component as thickening agent. The exact cause of this effect is not known. The use of metal oxides to prevent the dental pastes from bleeding is a subject-matter of the invention.

Oxides of group 4 metals are preferably used in a quantity of from 0.05 to 3 wt.-%, particularly preferably 0.5 to 3 wt.-% and quite particularly preferably 0.5 to 1.5 wt.-%.

### Additives and Auxiliaries

In addition to the named components, the prophy pastes according to the invention can advantageously contain one or more auxiliaries, such as for example fluoride-releasing additives, flavoring agents, sweetening agents, dyes, stabilizers and agents which prevent the growth of microorganisms.

Fluoride compounds are preferred as fluoride-releasing additives, in particular sodium fluoride, potassium fluoride, ammonium fluoride, ammonium bifluoride, sodium monofluorophosphate, potassium monofluorophosphate, calcium fluoride, salts of tetra- or hexafluoro anions, such as e.g. ammonium hexafluorosilicate, magnesium hexafluorosilicate, potassium hexafluorophosphate, ammonium hexafluorotitanate, ammonium tetrafluorotitanate, ammonium hexafluoroaluminate, zirconium fluoride, tetra-n-butylammonium dihydrogentrifluoride (TBAF-3), rubidium fluoride, caesium fluoride, potassium bifluoride (KHF₂), silver(I) fluoride (AgF), tin(II) fluoride (SnF₂), olaflur and dectaflur. The following are particularly preferred: sodium fluoride, potassium fluoride, sodium monofluorophosphate, tin(II) fluoride (SnF₂), ammonium fluoride, olaflur and dectaflur.

The fluoride component is preferably used in a concentration of from 0 to 20,000 ppm, particularly preferably from 100 to 5000 ppm and quite particularly preferably from 500 to 1500 ppm. These figures relate to the quantity of fluoride ions in the overall composition.

Flavoring agents give the prophy paste the desired taste. Preferred flavoring agents are natural or artificial compositions with a peppermint, strawberry, raspberry, banana, lemon, bubblegum or apple flavor. The prophy pastes can contain one or more flavoring agents.

The prophy pastes according to the invention can furthermore contain one or more sweetening agents. Preferred sweetening agents are acesulfame, aspartame, cyclamate, neohesperidin, saccharin, sucralose, stevioside. Sugar alcohols such as xylitol, erythritol and sorbitol can also be used as sweetening agents, but relatively large quantities of them are required.

### Water Activity

The dental prophy pastes according to the invention are characterized in that they retain their smooth consistency even under unfavorable ambient climatic conditions and do not dry out even when they are not packaged in a vapor-tight manner. This is achieved by matching the type and quantity of the components used to each other in an advantageous manner in the pastes according to the invention. It has been found that the prophy pastes have particularly good properties when the type and quantities of the components are chosen such that the pastes have a water activity in the range of from 0.5 to 0.7, preferably 0.5 to 0.65 and particularly preferably from 0.55 to 0.65, in each case measured at a temperature of 25° C.

The water activity (aw) at a given temperature is defined as the ratio of the water vapor partial pressure of the formulation to the saturation vapor pressure of pure water. The water activity of the pastes is determined by placing 3 g of the prophy paste in a hermetically sealable measuring chamber with a volume of 15 ml and then measuring the humidity being established in the enclosed residual air. The humidity is measured in % relative humidity (RH) and is related to the aw value as follows: a_{w}=RH/100. The measurement is preferably effected with a commercially available measuring device, e.g. with the LabTouch-aw advanced model (from Novasina AG, Neuheimstrasse 12, CH-8853 Lachen).

Setting the water activity makes it possible to provide reproducible pastes with the desired properties. The water activity can be set particularly well by varying the quantities of the humectant(s) and of the water. These quantities are preferably chosen such that the water content lies in the range of from 20 to 30 wt.-%, preferably 22 to 29 wt.-% and particularly preferably 24 to 28 wt.-%, relative to the total quantity of water and humectant.

### Disc Consistency

The pastes according to the invention are characterized by a creamy, smooth consistency, which guarantees that they can be dispensed easily from tubes and have a good spreadability. They can therefore be easily picked up by dental rubber cups or polishing brushes and distributed on the teeth. Rubber cups are rotating dental instruments in the form of a hollow cylinder. They are used together with a rotating handpiece to clean or polish the teeth. They are typically applied with a contact pressure of approx. 2 N and a rotational speed of 2000-10,000 rotations per minute.

The disc consistency is used as a measure of the consistency of the pastes. To measure the disc consistency, a defined quantity (0.70±0.01 g) of the sample brought to the correct temperature (23±2° C.) is compressed between two square glass plates (thickness approx. 20 0.7 mm, edge length approx. 50 mm) with a defined test load (120 g) at 23 ±2° C. for three minutes. The diameter of the compressed sample (“disc diameter”) is then measured precisely to 0.01 mm. The diameter in mm corresponds to the disc consistency.

The pastes according to the invention preferably have a disc consistency of from 20 to 40 mm, particularly preferably from 25 to 38 mm and quite particularly preferably from 28 to 35 mm.

### Flow Properties

The prophy pastes according to the invention are characterized in addition by a good creeping strength, with the result that they do not flow out of dispensers, such as for example finger clips. Finger clips are small vessels with a volume of approx. 2 ml, which can be worn on the fingers with a holder.

For the measurement of the flow properties, a defined quantity (1.5±0.05 g) of the sample brought to the correct temperature (23±2° C.) is distributed in a circle (circle diameter approx. 2 cm) on a mixing block (synthetic paper based on HDPE, e.g. Polyart®, from Arjobex, Boulogne-Billancourt, France). The mixing block with the sample is then allowed to rest horizontally for 15±0.5 minutes at 23±2° C. and then positioned vertically. After 15±0.5 minutes how many mm the front of the paste has moved downwards is measured. The pastes according to the invention preferably flow less than 10 mm, particularly preferably less than 3 mm and quite particularly preferably less than 1 mm.

According to the invention, prophy pastes with the following composition are particularly preferred:
1 to 20 wt.-%, preferably 1.5 to 16 wt.-% and particularly preferably 12 to 16 wt.-% water,
13 to 46 wt.-%, preferably 25 to 46 wt.-% and particularly preferably 30 to 40 wt.-% glycerol,
0 to 25 wt.-%, preferably 1 to 10 wt.-% and particularly preferably 1 to 5 wt.-% xylitol and/or sorbitol,
0 to 45 wt.-%, preferably 10 to 43 wt.-% and particularly preferably 12 to 22 wt.-% kaolin,
0 to 40 wt.-%, preferably 0 to 28 wt.-% and particularly preferably 20 to 28 wt.-% precipitated silica,
0.05 to 2 wt.-%, preferably 0.1 to 0.5 wt.-% and particularly preferably 0.1 to 0.2 wt.-% sodium lauryl sulfate, and
0.5 to 3 wt.-%, preferably 0.7 to 2.0 wt.-% and particularly preferably 1 to 1.3 wt.-% cetyl alcohol, stearyl alcohol or a mixture thereof,
wherein the total quantity of abrasives lies in the range of from 36 to 51 wt.-%.

According to a quite particularly preferred embodiment, the pastes additionally contain
0.5 to 3.0 wt.-%, preferably 0.7 to 2.5 wt.-%, particularly preferably 1.0 to 2.0 wt.-% Me^{IV}₂, in particular TiO₂, ZrO₂ or a combination thereof.

Prophy pastes with the following composition are further preferred:
12 to 16 wt.-% water,
30 to 40 wt.-% glycerol,
1 to 2 wt.-% xylitol,
1 to 3 wt.-% sorbitol,
at least one abrasive, selected from 12 to 22 wt.-% kaolin, 20 to 28 wt.-% precipitated silica, and 20 to 50 wt.-% pumice powder,
0.1 to 0.2 wt.-% sodium lauryl sulfate, and
1 to 1.3 wt.-% cetyl alcohol, stearyl alcohol or a mixture thereof,
1.0 to 2.0 wt.-% TiO₂, ZrO₂ or a combination thereof,
wherein the total quantity of abrasives lies in the range of from 36 to 51 wt.-%.

In all cases, the preferred and particularly preferred ranges named herein can be selected independently of each other.

### Preparation Methods

The prophy pastes according to the invention can be prepared in the simplest case by mixing the individual components with each other homogeneously.

Pastes which contain a fatty component as thickening agent are preferably prepared by first of all taking some of the water and of the humectant, preferably water and glycerol, and then suspending the abrasives therein. Color and taste components and further optional additives, such as e.g. the fluoride component, can also be incorporated into this suspension.

In a separate container, the fatty component is mixed with the remaining portion of water and humectant and the surfactant and heated until the fatty component is completely melted. The mixing is continued until there is a homogeneous mixture.

The mixture of fatty components is then added to the abrasive-containing suspension and mixed until homogeneous. In order to prevent the inclusion of air, the mixing is preferably effected under reduced pressure. A creamy-smooth, creep-stable paste is obtained.

The individual ingredients of the pastes according to the invention are preferably divided between the two mixtures such that the weight ratio of the abrasive-containing suspension to the mixture containing the fatty component lies in the range of from 95:5 to 50:50, wherein a ratio of 90:10 is particularly preferred.

The polishing and cleaning pastes according to the invention are particularly suitable for professional tooth cleaning, i.e. for the professional cleaning and polishing of natural teeth, of restorations and prosthetic fittings. The prophy pastes are suitable for removing dental plaque and extrinsic stains and minimize plaque accumulation and retention. For the gentle cleaning of natural teeth, and in particular of teeth which have been restored with relatively soft filling materials, it is important that the prophy pastes do not scratch the tooth surfaces. Conventional polishing pastes with a high cleaning power usually also have a high abrasiveness, which brings with it a high risk of scratching. Conversely, a low abrasiveness is typically accompanied by a lower cleaning power, which increases the time required for cleaning the teeth. Currently, in professional tooth cleaning, as a rule the teeth are first of all treated with a paste with high cleaning action and finally polished with a paste with low abrasiveness, in order to smooth the tooth surfaces. The prophy pastes according to the invention have an abrasiveness and cleaning action that is advantageous for dental purposes without leaving scratches on usual plastic filling materials and dental metals. They therefore make it possible to clean and polish tooth surfaces in only one operation.

In addition, their consistency and creeping strength guarantee that they do not run or drip off cleaning instruments such as brushes or rubber cups within a short time. They are thus easy to handle and make it possible to clean both natural tooth surfaces and restored tooth surfaces efficiently.

Also, the pastes do not dry out in a dry environment even if they are stored open or in insufficiently vapor-tight packaging. They do not bleed in a humid environment and do not start to run when they absorb moisture. They remain creamy-smooth and can be used in many climate zones.

The prophy pastes according to the invention can be marketed packed in tubes or cans. Single-dose packages which contain the quantity of prophy paste necessary for a single application, particularly preferably approx. 1-2 ml, are preferred. Plastic containers, in particular deep-drawn plastic containers, which are sealed with a plastic, aluminum or plastic/aluminum laminate film after filling, are preferably used as packaging. The plastic containers are preferably produced from polyethylene, polyamide, polypropylene, polystyrene, polyvinyl chloride or composite materials made of different plastics.

According to the present invention, teeth are preferably cleaned by a process comprising the following steps:
(1) Optionally removing calculus and/or plaque from the teeth, preferably with a scaler or an airflow device,
(2) applying a portion of the prophy paste according to the present invention to the surface of the teeth, preferably with a rubber cup or a rotary brush,
(3) polishing the teeth, preferably at a speed of 100-10 000 rpm of the cup or brush, more preferably 500-5000 and most preferably 2000 to 3000 rpm,
(4) optionally rinsing the teeth with water to remove paste residues.

In step (1), sodium bicarbonate and in particular glycine or erythritol are preferably used as abrasive powders for the airflow process.

In step (3) the teeth are polished preferably for 1-60 s, more preferably 2 — 10 s and most preferably 2-5 s per tooth.

According to a preferred embodiment of the present invention, steps (2) and (3) are each performed only once, i.e. only one prophy paste is used for cleaning the teeth. As mentioned above, it is standard practice in professional tooth cleaning to use two or more pastes. By using the prophy paste according to the invention, only one polishing step is needed, which represents a considerable simplification of the cleaning process and reduces the time required.

The method describe above is suitable for cleaning natural teeth and dental restorations. However, in contrast to natural teeth, many dental restorations and dental restoration materials, such as composites, resin dentures, dental alloys, titanium, implants and implant abutments, are rather soft. Therefore, for the cleaning of such restoration materials and restorations made therefrom, it is preferred to avoid the use of a scaler and airflow device in step (1), in particular airflow devices with highly abrasive powder such a sodium bicarbonate, and in step (2) a prophy paste is preferably used which contains only kaolin and/or silica as abrasives, as defined above. It has been found that these abrasives do not scratch soft dental restoration materials.

The invention is explained in more detail below by means of figures and examples.

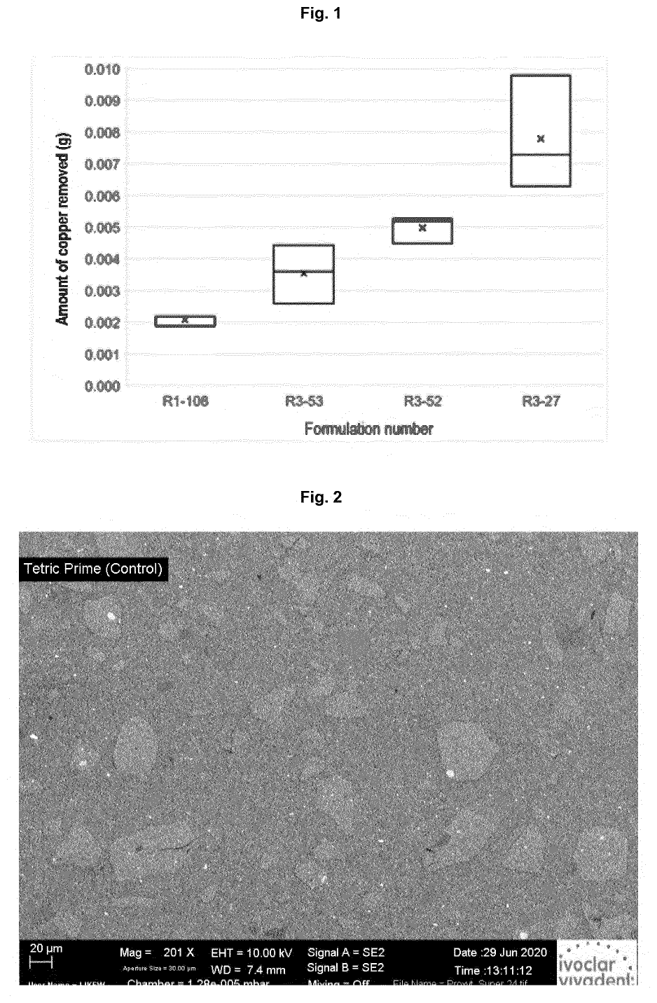

FIG. 1 shows the abrasiveness (cleaning power) of different prophy pastes. Paste R1-108 contains exclusively precipitated silica and paste R3-27 contains exclusively kaolin as abrasive. The remaining pastes contain mixtures of precipitated silica with kaolin with different particle sizes. It can be seen that the partial or complete replacement of the precipitated silica by kaolin increases the cleaning power of the pastes. The abrasiveness increases as the particle size of the kaolin used increases.

FIG. 2 shows the polished surface of a commercially available plastic filling material. The surface is smooth and has no visible scratches.

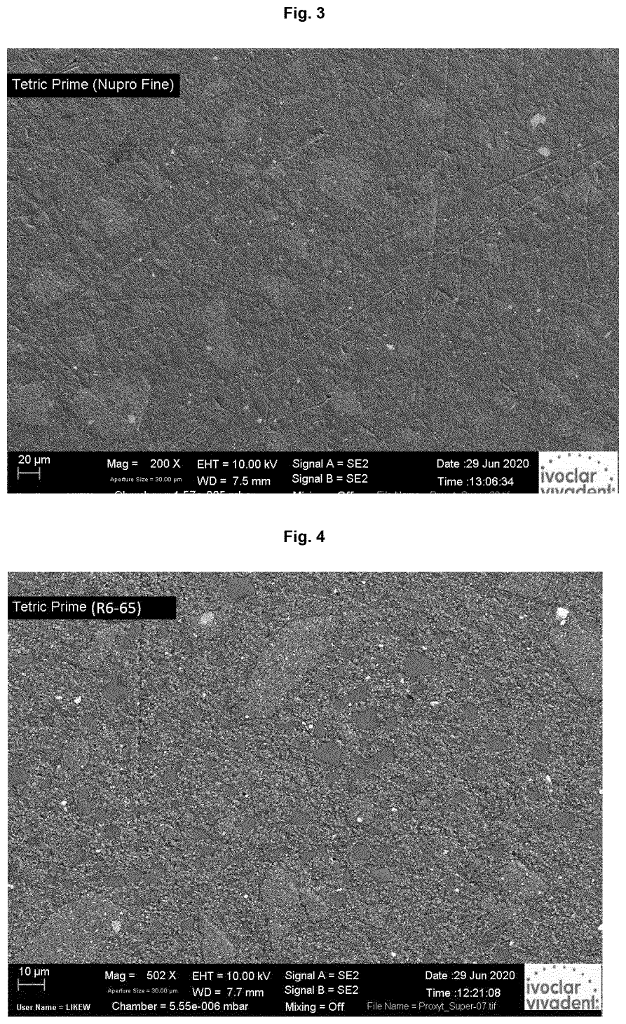

FIG. 3 shows the plastic filling material from FIG. 2 after the treatment with a conventional polishing paste. The paste left clear scratches on the surface.

FIG. 4 shows the plastic filling material from FIG. 2 after the treatment with a polishing paste according to the invention, which contains a mixture of kaolin and precipitated silica as abrasive. The plastic surface has no visible scratches.

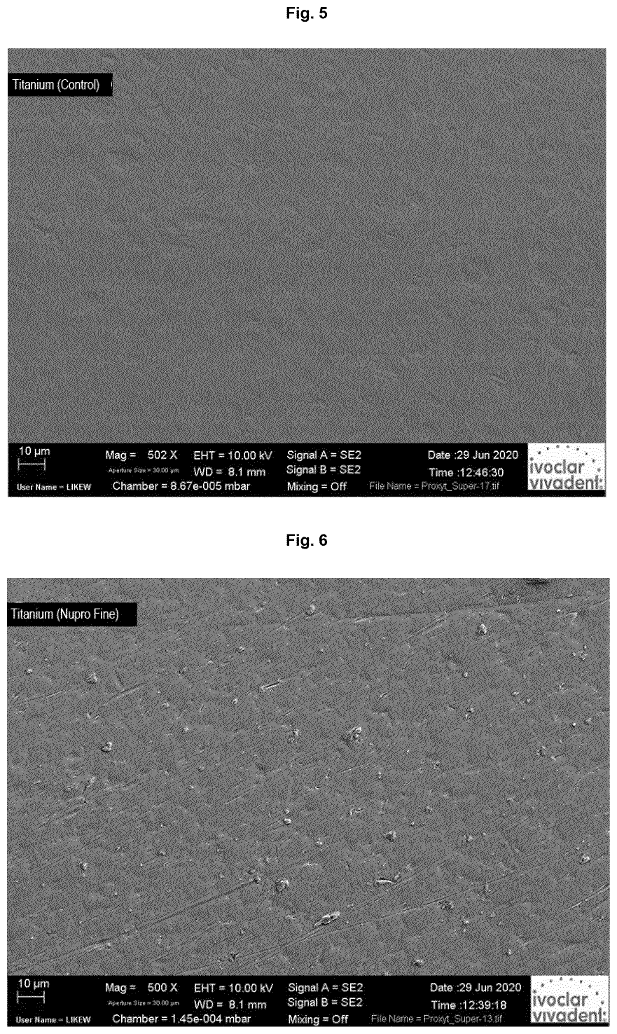

FIG. 5 shows a scanning electron micrograph of a polished titanium surface. The surface is smooth and has no scratches.

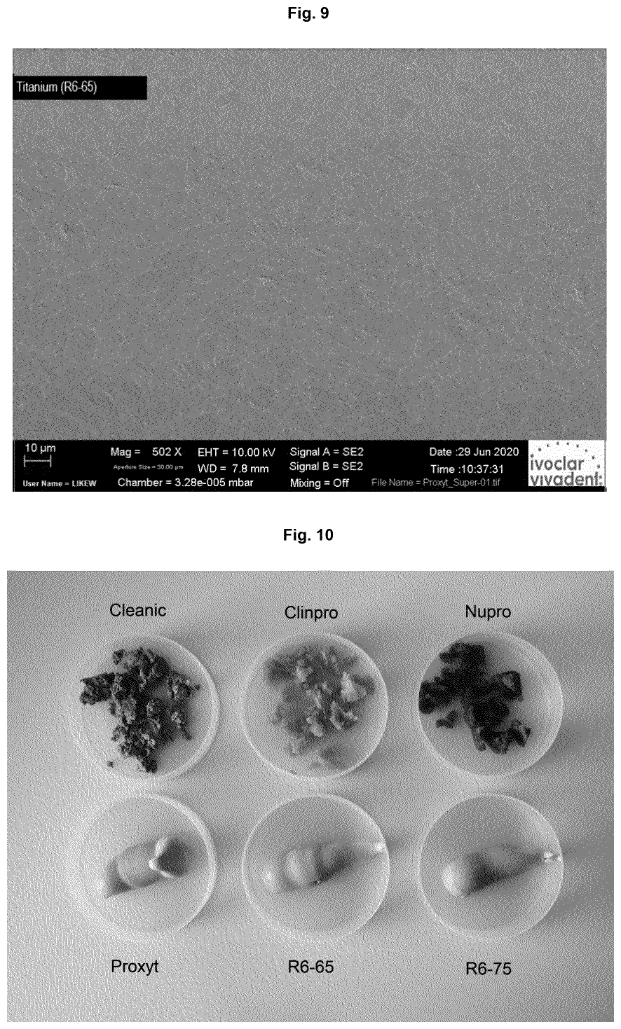

FIGS. 6 to 9 show scanning electron micrographs of titanium surfaces according to FIG. 5, which have been treated with conventional polishing pastes (FIGS. 6 to 8) or a polishing paste according to the invention (FIG. 9). The paste according to the invention left no scratches on the titanium surface; the surfaces treated with conventional pastes have clearly visible scratches.

FIG. 10 shows conventional prophy pastes and those according to the invention after removal from their containers. The product Proxyt RDA 7 fine and the pastes R6-65 and R6-75 according to the invention have a plastic, pasty consistency. The remaining products have a crumbly consistency and had to be taken out of the single-dose packaging with a spatula.

### EXAMPLES

### Example 1

### Preparation and Testing of Prophy Pastes

For the preparation of the pastes described in Table 1, most of the water and glycerol was placed in a mixing apparatus and the abrasives were suspended therein. The additives were then admixed. In a separate, heatable mixing apparatus, the fatty alcohol and the surfactant were mixed with the remaining portion of water and humectants and heated until the fatty alcohol melted completely. The mixing was continued until there was a homogeneous mixture. In the process, the fatty alcohol was emulsified in the polar phase by the surfactant. The mixture of the fatty alcohol was then added to the suspension from the first step and mixed under reduced pressure.

All formulations in Table 1 have a consistency and a flow behavior which is suitable for dental prophy pastes. Table 1 shows that pastes with a high water activity dry out quickly and then can no longer be used (R3-88). The water activity, disc consistency and flow properties were measured as described above. In the case of low water activity, the pastes behave hygroscopically and absorb water from the atmospheric humidity, which impairs their processing properties (R1-108, R3-89). In the case of pastes R1-108 and R3-89 the water absorption resulted in a liquid film forming on the surface of the material, which impairs the processing properties of the material. Pastes R3-52, R3-53, R3-27 only bleed a little, and in the case of pastes N1-152, R6-65, R6-75, R3-54, R3-80 and R6-73 the bleeding is further reduced again.

A comparison of pastes R3-89 and R3-54 shows that bleeding is particularly effectively prevented when the water activity lies above 0.5 and the paste contains a metal oxide.

| | | Example [wt .-%] | | | | | | | | | | | | | | |
|---|---|---|---|---|---|---|---|---|---|---|---|---|---|---|---|---|
| Ingredient/Property | | R1-108*) | N1-152 | R3-89*) | R3-88*) | R6-65 | R6-75 | R3-54 | R3-52 | R3-80 | R3-53 | R3-27 | R6-73 | R13-76 | R13-79 | R13-81 |
| Solvent | Water | 12.20 | 14.20 | 0.23 | 57.31 | 15.98 | 12.91 | 14.20 | 14.20 | 14.20 | 14.20 | 14.20 | 14.90 | 15.98 | 15.98 | 15.98 |
| Humectant | Glycerol | 45.22 | 43.09 | 57.08 | 0 | 39.09 | 31.28 | 43.11 | 43.11 | 43.09 | 43.11 | 43.11 | 36.19 | 39.09 | 39.09 | 39.09 |
| | Xylitol | 1 | 1 | 1 | 1 | 1 | 1 | 1 | 1 | 1 | 1 | 1 | 1 | 1 | 1 | 1 |
| | Sorbitol | 0 | 0 | 0 | 0 | 2.1 | 1.75 | 0 | 0 | 0 | 0 | 0 | 2.1 | 2.1 | 2.1 | 2.1 |
| Surfactant | Na lauryl sulfate ¹⁾ | 0.12 | 0.12 | 0.12 | 0.12 | 0.14 | 0.12 | 0.12 | 0.12 | 0.12 | 0.12 | 0.12 | 0.2 | 0 | 0 | 0 |
| | Dodecyltrimethylammonium chloride ¹⁷⁾ | 0 | 0 | 0 | 0 | 0 | 0 | 0 | 0 | 0 | 0 | 0 | 0 | 0.14 | 0 | 0 |
| | Hexadecylpyridiniumchloride monohydrate ¹⁹⁾ | 0 | 0 | 0 | 0 | 0 | 0 | 0 | 0 | 0 | 0 | 0 | 0 | 0 | 0.14 | 0 |
| | Olaflur¹⁸⁾ | 0 | 0 | 0 | 0 | 0 | 0 | 0 | 0 | 0 | 0 | 0 | 0 | 0 | 0 | 0.14 |
| Fatty component | Cetyl stearyl alcohol²⁾ | 1.06 | 1.06 | 1.06 | 1.06 | 1.27 | 1.06 | 1.06 | 1.06 | 1.06 | 1.06 | 1.06 | 1.27 | 1.27 | 1.27 | 1.27 |
| Metal | TiO₂¹⁵⁾ | 0.05 | 1 | 1 | 1 | 1 | 2 | 1 | 0 | 0 | 0 | 0.15 | 1 | 1 | 1 | 1 |
| oxide | ZrO₂ | 0 | 0 | 0 | 0 | 0 | 0 | 0 | 0 | 2 | 0 | 0 | 0 | 0 | 0 | 0 |
| Abrasive | Kaolin³⁾ | 0 | 12⁵⁾ | 12⁶⁾ | 12⁶⁾ | 12⁵⁾ | 0 | 12⁶⁾ | 12⁶⁾ | 12⁵⁾ | 12⁵⁾ | 40⁷⁾ | 0 | 12 | 12 | 12 |
| | Silica⁴⁾ | 40 | 27.15 | 27.15 | 27.15 | 27 | 0 | 27.15 | 28.15 | 26.15 | 28.15 | 0 | 23 | 27 | 27 | 27 |
| | Pumice stone powder | 0 | 0 | 0 | 0 | 0 | 49.48 | 0 | 0 | 0 | 0 | 0 | 20 | 0 | 0 | 0 |
| Additives | NaF | 0.12 | 0.12 | 0.12 | 0.12 | 0.12 | 0.12 | 0.12 | 0.12 | 0.12 | 0.12 | 0.12 | 0.12 | 0.12 | 0.12 | 0.12 |
| | Antifoaming agent | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| | Pigments | 0.01 | 0.04 | 0.02 | 0.02 | 0.08 | 0.06 | 0.02 | 0.02 | 0.04 | 0.02 | 0.02 | 0.08 | 0.08 | 0.08 | 0.08 |
| | Flavouring | 0.12 | 0.12 | 0.12 | 0.12 | 0.12 | 0.12 | 0.12 | 0.12 | 0.12 | 0.12 | 0.12 | 0.1 | 0.12 | 0.12 | 0.12 |
| Total | | 100 | 100 | 100 | 100 | 100 | 100 | 100 | 100 | 100 | 100 | 100 | 100 | 100 | 100 | 100 |
| Properties | Consistency [mm]⁸⁾ | 32 | 33 | 33 | 50 | 30 | 29 | 33 | 31 | 31 | 33 | 38 | 3' | 28 | 30 | 29 |
| | Flow [mm] | 0.0 | 0.0 | 6.1 | >20 | 0.0 | 0.0 | 0.5 | 0.0 | 0.56 | 0.0 | 0.0 | 0.0 | 0.0 | 0.5 | 5 |
| | Water activity | 0.48 | 0.56 | 0.13 | 0.97 | 0.60 | 0.6 | 0.56 | 0.56 | 0.56 | −¹⁶⁾ | 0.51 | 0.60 | 0.592 | 0.593 | 0.588 |
| | Usability after drying⁹⁾ | yes | yes | yes | no | yes | yes | yes | −¹⁶⁾ | −¹⁶⁾ | yes | yes | yes | yes | yes | yes |
| | Bleeding at 40° C., 75%^{10),14)} | +++ | + | +++ | 0¹²⁾ | + | 0 | 0 | ++ | + | ++ | ++ | 0 | ++ | ++ | ++ |
| | Bleeding at 25° C., 60%^{11),14)} | +++ | 0 | +¹³⁾ | 0¹²⁾ | 0 | 0 | −¹⁶⁾ | −¹⁶⁾ | −¹⁶⁾ | −¹⁶⁾ | −¹⁶⁾ | 0 | 0 | 0 | 0 |

*)Comparison example
¹⁾ CAS No. 151-21-3
²⁾CAS No. 67762-27-0
³⁾CAS No. 1332-58-7
⁴⁾Precipitated silica, average particle size (D50) 5.5-9.5 μm, CAS No. 112926-00-8
⁵⁾D50~3.9 μm
⁶⁾D50~6.6 μm
⁷⁾D50~8.0 μm
⁸⁾Disc consistency
⁹⁾Storage for 2 weeks at 23 ± 2° C. and 10% RH (desiccator over lithium chloride)
¹⁰⁾In the case of storage for 2 weeks at 40° C. and 75% RH (climate cabinet with temperature and humidity regulation);
¹¹⁾In the case of storage for 2 weeks at 25° C. and 60% RH (climate cabinet with temperature and humidity regulation);
¹²⁾Paste dried out
¹³⁾Paste became highly fluid
¹⁴⁾+++ = paste bleeds strongly; ++ = little; + = very little, 0 = does not bleed
¹⁵⁾Anatase; CAS No. 13463-67-7
¹⁶⁾Not measured
¹⁷⁾ CAS No. 112-00-5
¹⁸⁾2,2′-[[3-[(2-hydroxyethyl)octadecylamino]propyl]imino]bis-ethanol, dihydrofluoride
¹⁹⁾ CAS No. 6004-24-6

The water absorption and the water loss of the pastes were determined under extreme environmental conditions. For this, how much weight the pastes gained or lost in the case of dry storage at room temperature 23±2° C. (RT) at approx. 10% relative humidity (RH) or under tropical conditions (40° C., 75% RH) was quantified. The dry storage was carried out in a desiccator over lithium chloride. A digital humidity meter was placed in the desiccator, with the result that the drying out could be tracked. The samples were stored in the desiccator until an equilibrium moisture content of approx. 10% had been established. This was typically the case after 10 to 14 days. The storage under tropical conditions was effected in a climate cabinet with temperature and humidity regulation. The samples were stored under these conditions for approx. 2 weeks and, in this time, had reached a moisture content equilibrium with the environment. The measured values for two pastes are shown in Table 2 by way of example.

The prophy pastes named in Table 2 have a water activity of 0.56 and scarcely bleed even at a humidity of 75% RH. The differences in weight change found are negligible. Both pastes absorb approximately the same amount of moisture out of the environment and give off the same amount of moisture. Paste R3-54 is characterized in that, in comparison with paste R3-52, it does not bleed at all. This is to be attributed to the titanium dioxide content. Although the pastes became more solid when stored at room temperature (10% RH), they could still be picked up well with dental rubber cups and could be used without limitations. Even after storage at 40° C. (75% RH), the pastes remained smooth, could be picked up well in rubber cups and could be used without limitations.

TABLE 2
Weight changes of the pastes from Table 1 when stored
open under various environmental conditions

| Batch | R3-52 | R3-54 |
|---|---|---|
| Weight loss at RT, approx. 10% RH | 11.4 wt.-% | 11.3 wt.-% |
| Weight gain at 40° C., 75% RH | 14.5 wt.-% | 14.3 wt.-% |

### Example 2

### Measurement of the Abrasiveness of Prophy Pastes

For the determination of the abrasiveness (cleaning power) of the pastes, cylinders made of pure copper (diameter of 25 mm) were polished with the pastes and the amount of material removed was measured. Pure copper is softer than dental plastic filling materials and usual dental alloys. The method allows the abrasiveness to be measured in the laboratory under reproducible conditions. A commercially available grinding and polishing machine was used (Buehler EcoMet250 with AutoMet250 grinding head). A polishing cloth with a diameter of 10 inches (25.4 cm) (TexMet C type; Buehler, Lake Bluff, IL, USA Art. 40-1110) was coated with a thin layer (approx. 15 g) of the prophy paste to be tested using a spatula. The copper cylinders were then polished for 5 minutes using the grinding machine without water cooling under the following conditions:
Head speed: 30 rpm
Base speed: 100 rpm
Single grinding pressure: 20 N
Climb/conventional grinding: Climb

Prophy pastes without (paste R1-108*) and with (pastes R3-53, R3-52 and R3-27) kaolin were compared. The pastes contained kaolin with different particle sizes. The abrasion on the copper test pieces was quantified by determining the weight difference before and after the polishing using an analytical balance. FIG. 1 shows the abrasiveness of the prophy pastes tested. It can be seen that partially or completely replacing the precipitated silica with kaolin increases the abrasiveness and thus the cleaning power of prophy pastes. The abrasiveness increases as the average particle size of the kaolin used increases.

### Example 3

### Determination of the Scratch Properties of Prophy Pastes on Plastic Filling Materials

The scratch behavior of a polishing paste according to the invention was compared with that of a product customary in the market. For this, a composite filling material (Tetric Prime of color A3; Ivoclar Vivadent AG, Schaan, Liechtenstein) was polymerized in accordance with the manufacturer's instructions, then ground, and then polished with 4000 grit silicon carbide paper with water cooling. FIG. 2 shows the polished surface of the plastic filling material. A smooth surface without scratches is visible.

The smooth surface was then polished with a commercially available polishing paste (Nupro Fine, Dentsply Professional, York PA, USA, Article Number 801232, Lot: 00022171) for 10 s and with a 2 N grinding pressure at 3000 revolutions per minute using a dental rubber cup. The product left clear scratches on the surface (FIG. 3).

For comparison, the smooth surface of the composite filling material was treated with the paste R6-65 according to the invention in the manner described. FIG. 4 shows that the polishing paste according to the invention which contains a mixture of a precipitated silica and kaolin as abrasive did not lead to scratching of the plastic surface.

The results of Example 2 and Example 3 show that prophy pastes according to the invention have an abrasiveness and cleaning action that is advantageous for dental purposes, without scratching usual plastic filling materials. The kaolin-containing pastes were comparably gentle as precipitated silica but achieved a much better cleaning action, with the result that the cleaning procedure took up much less time.

### Example 4

### Scratch Properties of Prophy Pastes on Dental Titanium

Grade 5 titanium is used for the production of implant abutments. These parts can also be fitted in the oral cavity and must be cleaned. Since many metals, and in particular grade 5 titanium, are even more susceptible to scratching than dental plastic filling materials, a particularly gentle approach is required here in order not to damage the surface.

To compare the scratch properties of polishing pastes according to the invention and commercially available materials, grade V titanium was polished. For this, round titanium discs (19 mm diameter, 2.25 mm thickness) were embedded in GTS polystyrene casting resin. Then, the discs were wet-ground with 4000 grit silicon carbide paper and then polished to a high gloss with a polishing cloth (Chemomet Polishing Cloth Art. 40-7920, Buehler, LakeBuff, IL, USA) and a series of polishing agents (1. Buehler MasterPrep Polishing Suspension 0.05 μm, Art. 40-6377-032; 2. Buehler MasterMet Colloidal Silica Suspension, Art. 40-6370-006; 3. Buehler MasterMet 2 Non-crystallizing colloidal silica polishing suspension, Art. 40-6380-006). In each step, the discs were polished for 10 min at 100 rpm and with a grinding pressure of 20 N, and only 15 N in the last step. FIG. 5 shows a scanning electron micrograph of the polished surface. The surface is smooth and has no scratches.

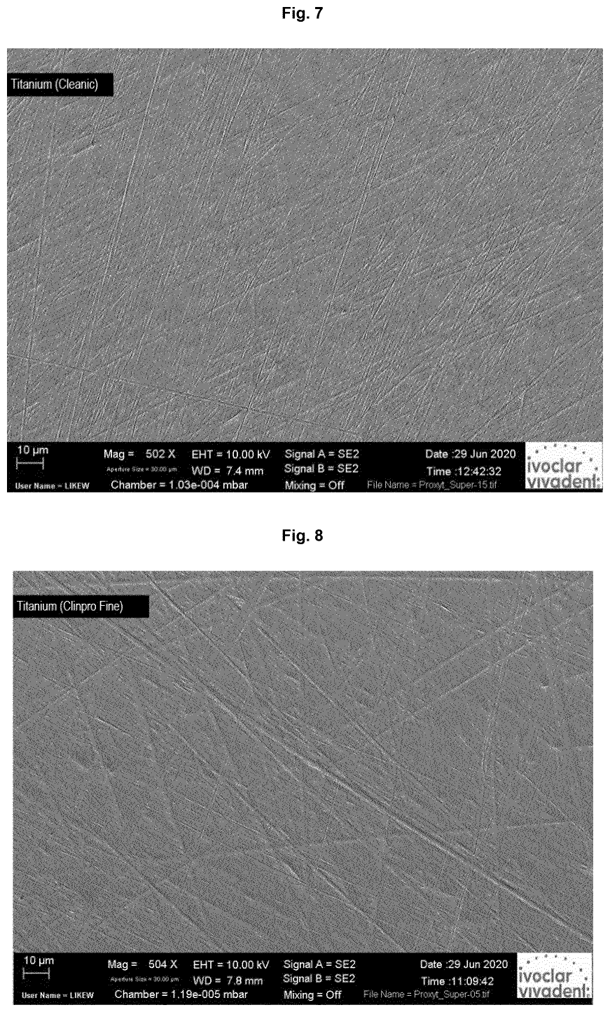

The titanium surfaces were then polished with the pastes to be tested for 10 s and with 2 N grinding pressure using a dental rubber cup at 3000 revolutions per minute. The following materials were used:
Nupro Fine (Dentsply Professional, York PA, USA, Article Number 801232, Lot: 00022171; FIG. 6)
Cleanic (KerrHawe SA, Bioggo, Switzerland, Article Number 3140, Lot: 7251052; FIG. 7)
Clinpro (3M Oral Care Solutions Division, St. Paul MN, USA, Article Number 12613, Lot: 091119A; FIG. 8)

FIGS. 6 to 8 show the surface of the titanium discs after the treatment. In all cases the products left clear scratches on the surface.

For comparison, the titanium surface was treated with the paste R6-65 according to the invention in the manner described. FIG. 9 shows that the polishing paste according to the invention did not leave any scratches at all on the titanium.

### Example 5

### Comparison of the Consistency of Pastes According to the Invention And Commercially Available Products

Most commercially available prophy pastes are sold in small individual packages and contain only a little water. They have a relatively hard consistency. Examples of this are the products Nupro (Dentsply Professional, York PA, USA), Clinpro (3M Oral Care Solutions Division, St. Paul MN, USA) and Cleanic (KerrHawe S A, Bioggo, Switzerland). The product Proxyt RDA 7 fine (Ivoclar Vivadent AG, Schaan, Liechtenstein) is supplied in tubes.

FIG. 10 shows the above-named products after removal from their containers. It can be seen that only Proxyt RDA 7 fine is a plastic pasty mass. The other products had to be taken out of the single-dose packaging with a spatula. They are difficult to pick up with rubber cups.

The prophy pastes R6-65 (Lot: YM1278) and R6-75 according to the invention are also shown for comparison. Like the product Proxyt RDA 7 fine, pastes R6-65 and R6-75 have a creamy-smooth consistency and can be stored both in tubes and in small single-dose packages. They can be easily dispensed from a tube.

The pastes were stored under various conditions (cf. Table 3). The properties of the paste R6-65 according to the invention scarcely changed. It remained homogeneous and plastic over a large humidity range (from 10% RH to 75% RH) and can therefore also be kept in individual packages without problems. Even in the opened package it stays creamy-smooth over a longer period.

The product Proxyt RDA 7 fine was also pasty at the start, but dried out relatively quickly. The remaining pastes retained their crumbly consistency and were comparatively difficult to process.

TABLE 3
Properties of prophy pastes according to the
invention compared with market products

| | | | Proxyt | | | |
|---|---|---|---|---|---|---|
| | | | RDA 7 | | | |
| Property | R6-65 | R6-75 | fine¹⁾ | Nupro²⁾ | Clinpro³⁾ | Cleanic⁴⁾ |
| Disc | 30 | 29 | 32 | Not | Not | Not |
| consistency | | | | measur- | measur- | measur- |
| (mm) | | | | able | able | able |
| Flow (mm) | 0.0 | 0.0 | 4.0 | Not | Not | Not |
| | | | | measur- | measur- | measur- |
| | | | | able | able | able |
| Consistency in | Creamy- | Creamy- | Pasty | Solid | Solid | Solid |
| packaging | smooth | smooth | | | | |
| Behavior when | Soft | Soft | Tough | Crumbly | Crumbly | Crumbly |
| dispensed | plastic | plastic | plastic | | | |
| Stored open | Smooth | Smooth | Dried | —⁵⁾ | —⁵⁾ | —⁵⁾ |
| at 23° C., | | | out | | | |
| 10% RH | | | | | | |
| Stored open | Creamy- | Creamy- | Dried | —⁵⁾ | —⁵⁾ | —⁵⁾ |
| at 25° C., | smooth | smooth | out | | | |
| 60% RH | | | | | | |
| Stored open | Creamy- | Creamy- | Pasty | —⁵⁾ | —⁵⁾ | —⁵⁾ |
| at 40° C., | smooth | smooth | | | | |
| 75% RH | | | | | | |

¹⁾Proxyt RDA 7—fine (Ivoclar Vivadent AG, Schaan, Liechtenstein, Article Number 579866, Lot: Y51058)
²⁾Nupro (Dentsply Professional, York PA, USA, Article Number 801232, Lot: 00022171)
³⁾Clinpro (3M Oral Care Solutions Division, St. Paul MN, USA, Article Number 12613, Lot: 091119A)
⁴⁾Cleanic (KerrHawe SA, Bioggo, Switzerland, Article Number 3140, Lot: 7251052)
⁵⁾Not measured

1. A dental polishing and cleaning paste, which comprises
1 to 30 wt.-% water,
25 to 70 wt.-% of at least one humectant,
25 to 60 wt.-% of at least one abrasive and
0.05 to 2 wt.-% of at least one surfactant,
in each case relative to the total mass of the polishing and cleaning paste.

2. The polishing and cleaning paste according to claim 1, which comprises as the at least one abrasive 0 to 15 wt.-% kaolin and/or 0 to 50 wt.-% pumice powder and/or 0 to 30 wt.-% silica, in each case relative to the total mass of the polishing and cleaning paste, wherein the total quantity of the abrasive lies in the range named in claim 1.

3. The polishing and cleaning paste according to claim 2, in which the kaolin powder has an average particle size (D50) of from 1 to 20 μm, and/or a particle size (D90) of less than 30 μm.

4. The polishing and cleaning paste according to claim 2, in which the kaolin powder has an average particle size (D50) of from 3 to 10 μm and/or a particle size (D90) of less than 15 μm.

5. The polishing and cleaning paste according to claim 2, which comprises at least 10 wt.-% of kaolin, relative to the total quantity of abrasive.

6. The polishing and cleaning paste according to claim 5, which comprises 25 to 35 wt.-% of kaolin, relative to the total quantity of abrasive.

7. The polishing and cleaning paste according to claim 1, which comprises as the at least one abrasive a mixture of silica and kaolin powder and no further abrasives.

8. The polishing and cleaning paste according to claim 1, which additionally comprises 0.5 to 3 wt.-% of at least one fatty component, relative to the total mass of the polishing and cleaning paste.

9. The polishing and cleaning paste according to claim 1, which additionally comprises 1 to 1.3 wt.-% of at least one fatty component, relative to the total mass of the polishing and cleaning paste.

10. The polishing and cleaning paste according to claim 8, which comprises as the at least one fatty component a fatty alcohol with a chain length of from 12 to 22 carbon atoms.

11. The polishing and cleaning paste according to claim 10, which comprises as the at least one fatty component a fatty alcohol with a chain length of from 16 to 18 carbon atoms.

12. The polishing and cleaning paste according to claim 1, which additionally comprises 0.05 to 3 wt.-% of at least one metal oxide, relative to the total mass of the polishing and cleaning paste.

13. The polishing and cleaning paste according to claim 1, which additionally comprises 0.5 to 1.5 wt.-% of at least one metal oxide, relative to the total mass of the polishing and cleaning paste.

14. The polishing and cleaning paste according to claim 12, wherein the at least one metal oxide is selected from titanium dioxide, zirconium oxide and anatase.

15. The polishing and cleaning paste according to claim 1, which additionally comprises at least one additive, which is selected from fluoride sources, sweetening agents, flavoring agents and mixtures thereof.

16. The polishing and cleaning paste according to claim 1, which has a water activity of from 0.5 to 0.70, measured at a temperature of 25° C.

17. The polishing and cleaning paste according to claim 1, which has a water activity of from 0.55 to 0.65, measured at a temperature of 25° C.

18. The polishing and cleaning paste according to claim 1, which has a disc consistency of from 20 to 40 mm.

19. The polishing and cleaning paste according to claim 1, which has a disc consistency of from 28 to 35 mm.

20. The polishing and cleaning paste according to claim 1, which flows less than 10 mm, when 1.5±0.05 g of the sample brought to a temperature of 23±2° C. is distributed in a circle on a mixing block (circle diameter approx. 2 cm), the mixing block with the sample is then allowed to rest horizontally for 15±0.5 minutes at 23±2° C. and then positioned vertically and then, after 15±0.5 minutes, a number of mm the front of the paste has moved downwards is measured.

21. The polishing and cleaning paste according to claim 1, in which a quantity of the at least one humectant(s) is chosen such that the weight ratio of water to the total quantity of humectant(s) lies in the range of from 0.05 to 0.6.

22. The polishing and cleaning paste according to claim 1, which comprises
1 to 20 wt.-% water,
13 to 46 wt.-% glycerol,
0 to 25 wt.-% xylitol and/or sorbitol,
0 to 45 wt.-% kaolin,
0 to 40 wt.-% precipitated silica,
0.05 to 2 wt.-% of at least one cationic, anionic, and/or nonionic surfactant,
0.5 to 3 wt.-% cetyl alcohol, stearyl alcohol or a mixture thereof, and
optionally 0.5 to 3.0 wt.-% Me^{IV}O₂,
in each case relative to the total mass of the polishing and cleaning paste, wherein the total quantity of abrasives lies in the range of from 36 to 51 wt.-%.

23. The polishing and cleaning paste according to claim 1, wherein the at least one surfactant is selected from the group consisting of sodium lauryl sulfate, lauryl glucoside, sodium lauroyl sacrosinate, hexadecylpyridinium chloride, and dodecyltrimethylammonium chloride.

24. A process for cleaning and polishing of teeth comprising the following steps:
(1) optionally removing calculus and/or plaque from the teeth,
(2) applying a portion of the polishing paste according to claim 1 to a surface of the teeth,
(3) polishing the teeth for 1-60 s per tooth, and
(4) optionally rinsing the teeth with water to remove paste residues.

25. (canceled)
